(19) United States
(12) Patent Application Publication
(10) Pub. No.: US 2023/0138432 A1
(43) Pub. Date: May 4, 2023

(54) PORTABLE CIRCULATORY SHOCK DETECTING DEVICE

(71) Applicant: Tzong-Yann LEE, Taipei City (JP)

(72) Inventors: Tzong-Yann LEE, Taipei City (TW); Chi-Huang CHEN, Taipei City (TW); Hsin-Hui WANG, Taipei City (TW)

(21) Appl. No.: 17/515,752

(22) Filed: Nov. 1, 2021

(51) Int. Cl. A61B 5/00; A61B 5/024; A61B 5/0205; A61B 5/113; A61B 5/08

(52) U.S. Cl. CPC A61B 5/7275; A61B 5/024; A61B 5/02055; A61B 5/113; A61B 5/746; A61B 5/7203; A61B 5/0816; A61B 5/02438; A61B 5/002; A61B 2562/0219

(57) ABSTRACT

A portable circulatory shock detecting device is disclosed. In a measurement device, a body surface vibration data of a subject is sensed by a vibration-sensing module and transmitted to an electronic device by a communication interface. A heartbeat acquisition process is executed to obtain a heartbeat vibration data of the subject by a monitoring module of the electronic device, a circulatory shock detection process is executed to generate a heartbeat evaluation based on the heartbeat vibration data, and a circulatory shock alarm is outputted while the heartbeat evaluation is assessed as a circulatory shock risk.

### BACKGROUND OF THE DISCLOSURE

### Technical Field

The technical field relates to a portable device, and more particularly related to a portable circulatory shock detecting device.

### Description of Related Art

Circulatory shock is defined as a state of cellular and tissue hypoxia due to reduced oxygen delivery and/or increased oxygen consumption or inadequate oxygen utilization.

The effects of shock are initially reversible but can rapidly become irreversible, resulting in multi-organ failure, cardiac arrest, and death. Thus, when a patient is suspected of having shock, it is important that the clinician rapidly identify the etiology so that appropriate therapy can be administered to prevent multi-organ failure, cardiac arrest, and death.

Shock most commonly presents with hypotension. Hypotension occurs in the majority of patients with shock. Hypotension may be absolute (e.g., systolic blood pressure<90 mmHg; mean arterial pressure<65 mmHg), relative (e.g., a drop in systolic blood pressure>40 mmHg), orthostatic (>20 mmHg fall in systolic pressure or >10 mmHg fall in diastolic pressure with standing), or profound (e.g., vasopressor-dependent).

An instrument called a sphygmomanometer is used to take blood pressure readings. The blood pressure measurement by using the sphygmomanometer needs to spend a few minutes each time. The process is also uncomfortable because the inflatable cuff squeezes the patient's arm during measurement. Usually, only three or four readings are taken daily at home or during admission. Therefore, sphygmomanometer is unable to achieve continuous monitoring for blood pressure and the occurrence of circulatory shock.

In the intensive care unit, an arterial catheter is placed into an artery in the wrist, groin, or other location to measure blood pressure continuously and more accurately. Though arterial catheter could provide continuous monitoring of blood pressure and circulatory shock, it is an invasive procedure with high risk of infection, hematoma, acral ischemia.

Besides, neither the patient nor the monitoring device can move arbitrarily after arterial catheterization is done, which is inconvenient.

Thus, the existing circulatory shock monitoring device encounters the above-mentioned problems, a more convenient solution is needed.

### SUMMARY OF THE DISCLOSURE

The present disclosed example is direct to a portable circulatory shock detecting device having an ability to detect circulatory shock in a non-invasive manner and easily to be carried.

In one of the embodiments, a portable circulatory shock detecting device includes a measurement device and a monitoring module. The measurement device includes a vibration-sensing module and a communication module. The vibration-sensing module is used to sense a body surface vibration data of a subject. The communication module is electrically connected to the vibration-sensing module and used to transmit the body surface vibration data to an electronic device. The monitoring module is configured to control the electronic device to execute a heartbeat-acquiring process for the body surface vibration data to acquire a heartbeat vibration data corresponding to a heartbeat of the subject, execute a heartbeat-evaluating process for the heartbeat vibration data to generate a heartbeat evaluation of the subject, and output a circulatory shock alarm after the heartbeat evaluation is assessed as a circulatory shock risk.

The present disclosure may perform continuous circulatory shock detections anytime and anywhere and actively call for help while the subject is in circulatory shock, so that the subject may get rescued immediately and the survival probability of the subject may be improved.

### BRIEF DESCRIPTION OF THE DRAWINGS

The features of the present disclosure are believed to be novel are set forth with particularity in the appended claims. The present disclosure itself, however, may be best understood by reference to the following detailed description of the present disclosure which describes an exemplary embodiment of the present disclosure, taken in conjunction with the accompanying drawings, in which:

FIG. 1 is an architecture diagram of a portable circulatory shock detecting device according to one embodiment of the present disclosure;

FIG. 2 is an architecture diagram of a portable circulatory shock detecting device according to one embodiment of the present disclosure;

FIG. 3 is a flowchart of a method of circulatory shock monitoring according to one embodiment of the present disclosure;

FIG. 4 is a flowchart of respiratory evaluation according to one embodiment of the present disclosure;

FIG. 5 is a flowchart of heartbeat evaluation and respiratory evaluation according to one embodiment of the present disclosure;

FIG. 6 is a flowchart of data process according to one embodiment of the present disclosure;

FIG. 7 is a schematic view of a portable circulatory shock detecting device according to one embodiment of the present disclosure;

FIG. 8 is a schematic view of wearing a portable circulatory shock detecting device according to one embodiment of the present disclosure;

FIG. 9 is a schematic view of wearing a portable circulatory shock detecting device according to one embodiment of the present disclosure;

FIG. 10 is a schematic view of wearing a portable circulatory shock detecting device according to one embodiment of the present disclosure;

FIG. 11 is a schematic view of wearing a portable circulatory shock detecting device according to one embodiment of the present disclosure;

FIG. 12 is a waveform graph of body surface vibration data according to one embodiment of the present disclosure;

FIG. 13 is a waveform graph of heartbeat vibration data acquired from body surface vibration data of FIG. 12 according to one embodiment of the present disclosure;

FIG. 14 is a waveform graph of respiratory motion data acquired from body surface vibration data of FIG. 12 according to one embodiment of the present disclosure;

FIG. 15 is a waveform graph of heartbeat vibration data acquired from body surface vibration data of FIG. 12 according to one embodiment of the present disclosure; and

FIG. 16 is a waveform graph of respiratory motion data acquired from body surface vibration data of FIG. 12 according to one embodiment of the present disclosure.

### DETAILED DESCRIPTION OF THE EMBODIMENTS

The technical contents of this disclosure will become apparent with the detailed description of embodiments accompanied with the illustration of related drawings as follows. It is intended that the embodiments and drawings disclosed herein are to be considered illustrative rather than restrictive.

The present disclosure provides a portable circulatory shock detecting device being worn by a subject, such as patients, animals, or other biological subjects at circulatory shock risk. The portable circulatory shock detecting device enables to execute a circulatory shock detection on the subject continuously and instantly after the subject wears the portable circulatory shock detecting device.

The present disclosure enables to use a vibration-sensing module to measure a body surface vibration data induced by heartbeat of the subject, execute an analysis process (such as a frequency analysis, an amplitude analysis, a waveform analysis, etc.) for the body surface vibration data to obtain a heartbeat vibration data related to heartbeat intensity, and detect whether the circulatory shock occurs to the subject by monitoring the heartbeat vibration data.

The present disclosure executes the circulatory shock monitoring by sensing the vibration on the body surface, and the vibration on the body surface is propagable by the external medium. As a result, the portable circulatory shock detecting device of the present disclosure may be worn in a convenient and non-invasive manner, such as directly contacting the subject's skin (such as being pasted on the subject's skin by a patch structure, a fixed-line, or other assistive device) or indirectly contacting the subject's skin (such as being hanged on the clothing or the quilt and sensing the vibration across the clothing or the quilt).

In another embodiment, the present disclosure discloses that different analysis processes are executed based on the same body surface vibration data to respectively measure a heartbeat status (such as a heartbeat rate or a heart regularity) and a respiratory status (such as a respiratory rate or a respiratory regularity).

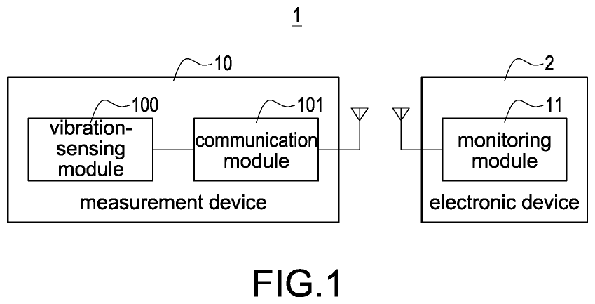

Please refer to FIG. 1, FIG. 1 is an architecture diagram of a portable circulatory shock detecting device according to one embodiment of the present disclosure. A portable circulatory shock detecting device 1 of this embodiment includes a measurement device 10 and a monitoring module 11. The measurement device 10 includes a vibration-sensing module 100 and a communication module 101.

The vibration-sensing module 100 may be one or more accelerometer(s), force sensor(s) or other types of vibration sensor(s). The above accelerometer may be a single axis accelerometer configured to sense and generate a single axis sensed acceleration data or a multi-axis accelerometer configured to sense and generate a multi-axis sensed acceleration data, but is not intended to limit the scope of the present disclosure. The vibration-sensing module 100 is used to sense the vibration induced by heartbeat at a specific part (such as the chest or the abdomen) of the body surface of the subject, and generate the body surface vibration data correspondingly.

The communication module 101, such as a Bluetooth network module, a ZigBee network module, a Wi-Fi network module, an infrared network module, an RFID module, an NFC module, or other types of wireless transmitter, is used to connect to an electronic device 2, and transfer data (such as the body surface vibration data or a body temperature data described later) to the electronic device 2.

The monitoring module 11 may be a hardware controller such as an MCU, a processor, or other types of the hardware controller, or a control software such as an application program, firmware, or other types of control software. The monitoring module 11 is arranged/installed in the electronic device 2 and used to control the electronic device 2. More specific, the monitoring module 11 may control the electronic device 2 to receive the above-mentioned body surface vibration data from the communication module 101, execute a heartbeat-evaluating process for the body surface vibration data to obtain a heartbeat evaluation, and determine whether the heartbeat evaluation is assessed as a circulatory shock risk.

In one of the embodiments, the electronic device 2 is specially dedicated to the measurement device 10. The monitoring module 11 may be a controller, a firmware, a dedicated chip, etc. of the electronic device 2.

In one of the embodiments, the electronic device 2 may be a smartphone, a smart wear, a tablet, or other portable device of the subject. The monitoring module 11 may be an application program stored and installed in the electronic device 2.

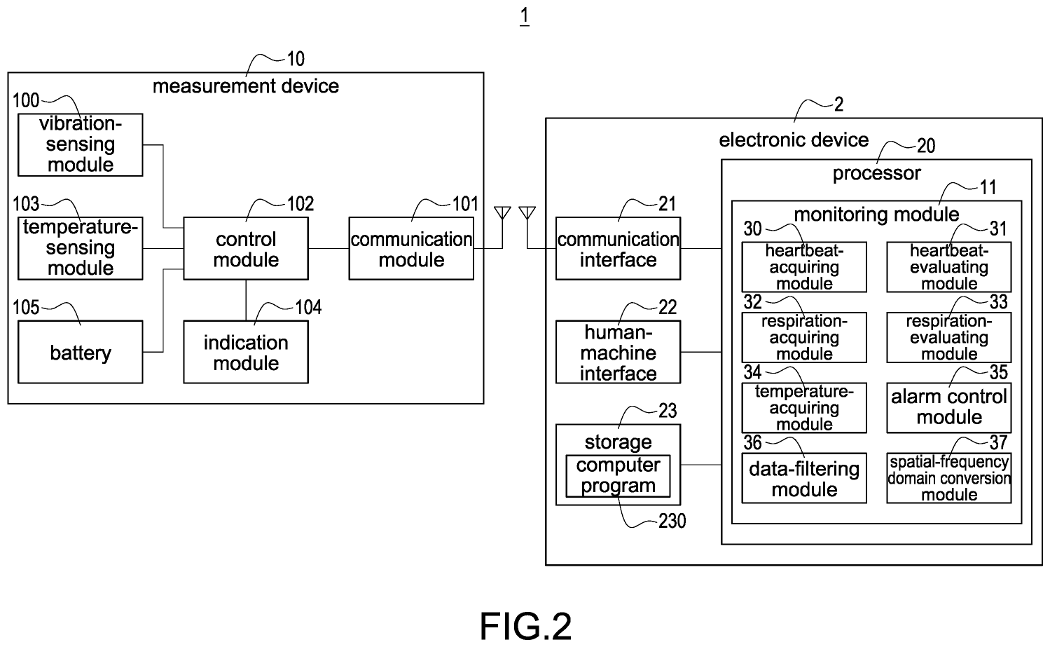

Please refer to FIG. 2, FIG. 2 is an architecture diagram of a portable circulatory shock detecting device according to one embodiment of the present disclosure. In comparison with the embodiment shown in FIG. 1, the measurement device 10 shown in FIG. 2 further includes at least one of a control module 102, a temperature-sensing module 103, an indication module 104 and a battery 105.

The control module 102, such as a System on Chip, a micro-controller, or a processor, is connected to each module of the measurement device 10, and used to control the operations and executions of the data/signal process of each module.

The temperature-sensing module 103, such as an infrared thermometer or a contact thermometer, is used to measure the body temperature of the subject, and generate the body temperature data correspondingly.

The indication module 104, such as a dot matrix display, an indicator light, a buzzer, or other low power output interface, is used to indicate the status of the measurement device 10 by screen picture, light and/or sound. The status of the measurement device 10 may be operating statuses such as monitoring, pairing, pairing complete, low battery, charging complete, etc.

The battery 105 is used to store the electricity and provide the electricity to the measurement device 10 for operation. The battery 105 may be the rechargeable batteries (such as nickel-metal hydride batteries or lithium batteries) or the disposable batteries (such as mercury batteries or alkaline batteries).

In one of the embodiments, the electronic device 2 may include a processor 20 (such as a System on Chip, a micro-controller, or a CPU). The processor 20 is connected to each component of the electronic device 2, and used to control the operation of each component and execute the data/signal process.

In one of the embodiments, the electronic device 2 may include a communication interface 21. The communication interface 21 uses the communication technology (such as Bluetooth, Wi-Fi, or Zig-Bee) compatible with the communication technology used by the communication module 101, so that the communication interface 21 and the communication module 101 may communicate with each other.

In one of the embodiments, the communication module 101 may include a Bluetooth low energy interface, the communication interface 21 includes a compatible Bluetooth communication interface, so that the communication interface 21 and the communication module 101 may communicate with each other by Bluetooth technology. The communication module 101 with Bluetooth low energy may save the power consumption and extend the continuous measurement operating time of the measurement device 10.

In one of the embodiments, the communication interface 21 may be connected to another designated electronic device (referred to as a rescue device) through network. The above rescue device may be the smart device around the subject, a smart device of the emergency contact person pre-designated by the subject, or the emergency rescue unit (such as the control console of the hospital or the fire brigade), but this specific example is not intended to limit the scope of the present disclosure. The smart device around the subject may be installed the same/similar monitoring module 10 or use the same communication technology, so that the smart device may receive a circulatory shock alarm sent from the electronic device 2.

Thus, while circulatory shock or other risks of the subject is detected, the monitoring module 10 sends the circulatory shock alarm to the people around, designated emergency contact person or the emergency rescue unit instantly and automatically, so that the subject may get the real-time emergency rescue.

In one of the embodiments, the electronic device 2 may include a human-machine interface 22, such as an indicator light, a speaker, a microphone, buttons, a touch screen, or any combination of the above devices. The human-machine interface 22 is used to receive the user's operation and output information.

In one of the embodiments, the human-machine interface 22 may be used to output the circulatory shock alarm, such as playing a circulatory shock alarm sound through the speaker, displaying a circulatory shock alarm picture on the display screen, or showing a circulatory shock alarm light signal through the indicator light.

The storage 23, such as a RAM, a ROM, an EEPROM, a flash memory, other types of storage media or any combination of the above storage media, is used to store data.

In one of the embodiments, the processor 20 may include a monitoring module 11. The monitoring module 11 is configured to achieve the functions of heartbeat evaluating, respiration evaluating, automatic alarming, and/or data processing.

The monitoring module 11 may include modules 30-37 including a heartbeat-acquiring module 30, a heartbeat-evaluating module 31, a respiration-acquiring module 32, a respiration-evaluating module 33, a temperature-acquiring module 34, an alarm control module 35, a data-filtering module 36, and a spatial-frequency domain conversion module 37, those modules 30-37 are configured to implement different functions respectively.

The heartbeat-acquiring module 30 is used to acquire the heartbeat vibration data corresponding to heartbeat from the body surface vibration data.

The heartbeat-evaluating module 31 is used to execute a heartbeat-evaluating process based on the heartbeat vibration data to generate a heartbeat evaluation for a circulatory shock detection, a heartbeat stop detection, etc. Also, the heartbeat-evaluating module 31 enables to monitor other heartbeat-related information, such as a heartbeat rate or a heartbeat intensity.

The respiration-acquiring module 32 is used to acquire a respiratory motion data corresponding to respiration from the body surface vibration data.

The respiration-evaluating module 33 is used to execute a respiration-evaluating process for the respiratory motion data to acquire a respiratory evaluation.

The temperature-acquiring module 34 is used to acquire the body temperature data of the subject's body, and align the timing between the body temperature data and the body surface vibration data for analyzing the body temperature corresponding to each data value (such as sensing time point or sensing sequence) of the body surface vibration data.

The alarm control module 35 is used to output the circulatory shock alarm while a corresponding risk, such as the circulatory shock risk or a respiratory failure risk is detected.

The data-filtering module 36 is used to execute an analysis, a conversion, a filtering and other processing for the data (such as the body surface vibration data and the body temperature data).

The spatial-frequency domain conversion module 37 is used to converse the data, such as the body surface vibration data and the body temperature data, between the spatial domain and the frequency domain.

The above-mentioned monitoring module 11 and the above-mentioned modules 30-37 are connected to each other (such as by electrical connection or information link), and any one of the monitoring module 11 and the modules 30-37 may be a hardware module (such as an electronic circuit module, an integrated circuit module, an SoC, etc.), a software module (such as firmware, an operating system, or an application program) or a combination of the hardware module and the software module, this specific example is not intended to limit the scope of the present disclosure.

Please be noted that when one of the above-mentioned monitoring module 11 and the above-mentioned modules 30-37 is the software module such as firmware, an application programs or an operating system, the storage module 23 may include a non-transitory computer-readable media (not shown in figures). The non-transitory computer-readable media stores a computer program 230. The computer program 230 records computer-readable codes. When the processor 20 executes the above computer-readable codes, the functions of the monitoring module 11 and the modules 30-37 may be achieved correspondingly.

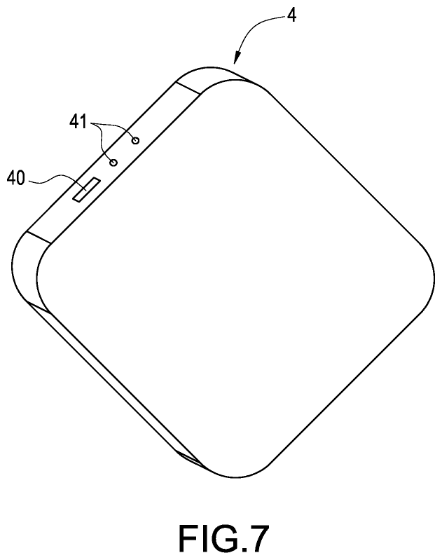

Please refer to FIG. 7, FIG. 7 is a schematic view of a portable circulatory shock detecting device according to one embodiment of the present disclosure. The measurement device 4 shown in FIG. 7 includes a flat outer case, and is suitable to be lean, placed, or worn on the body surface, such as the chest or the abdomen.

In one of the embodiments, the battery 105 of the measurement device 4 may include a rechargeable battery or include both a rechargeable battery and a disposable battery. The measurement device 4 may further include a charging connector 40 connected to the rechargeable battery and a related charging circuit (not shown in the figures). As a result, the cost of replacing a new disposable battery while low battery may be saved.

In one of the embodiments, the measurement device 4 may further include one or more indicator light(s) 41, such as one or more LEDs or multicolor LEDs, to indicate a current status of the measurement device 4.

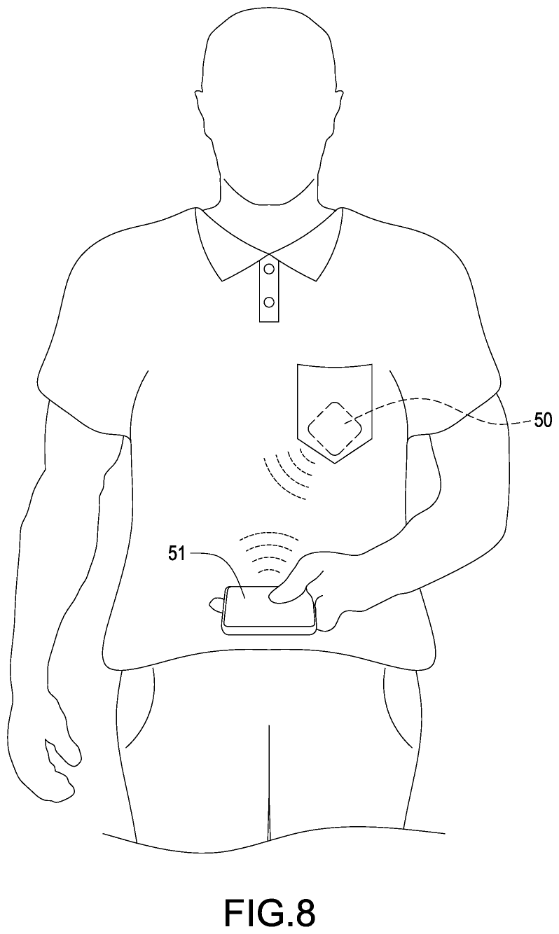

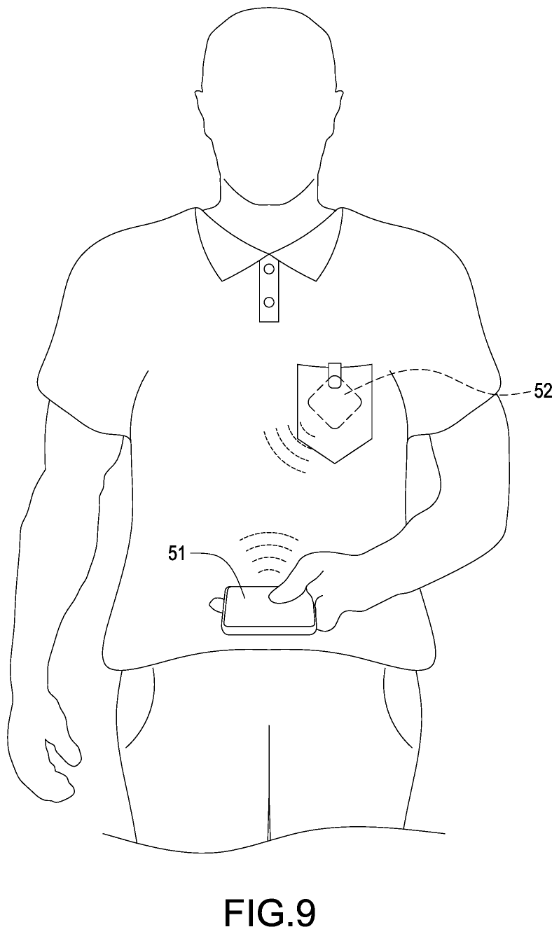

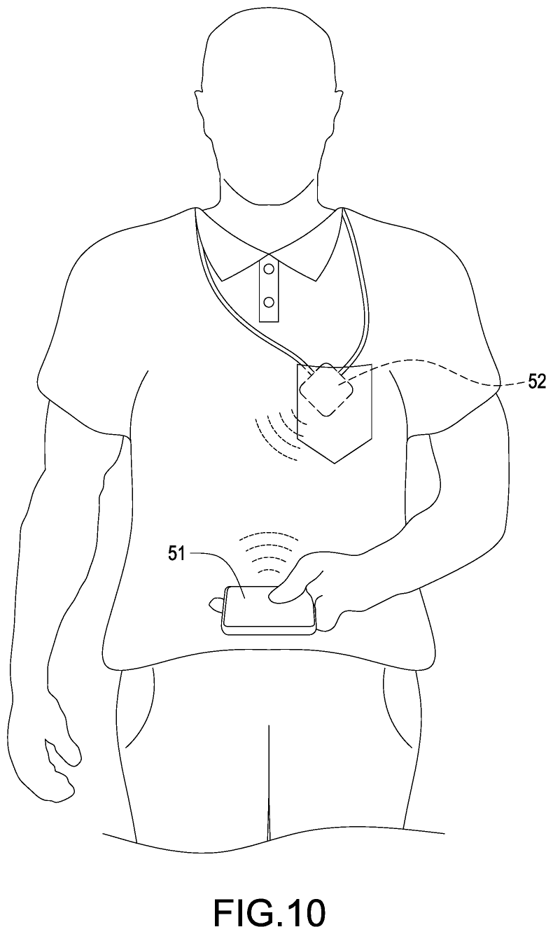

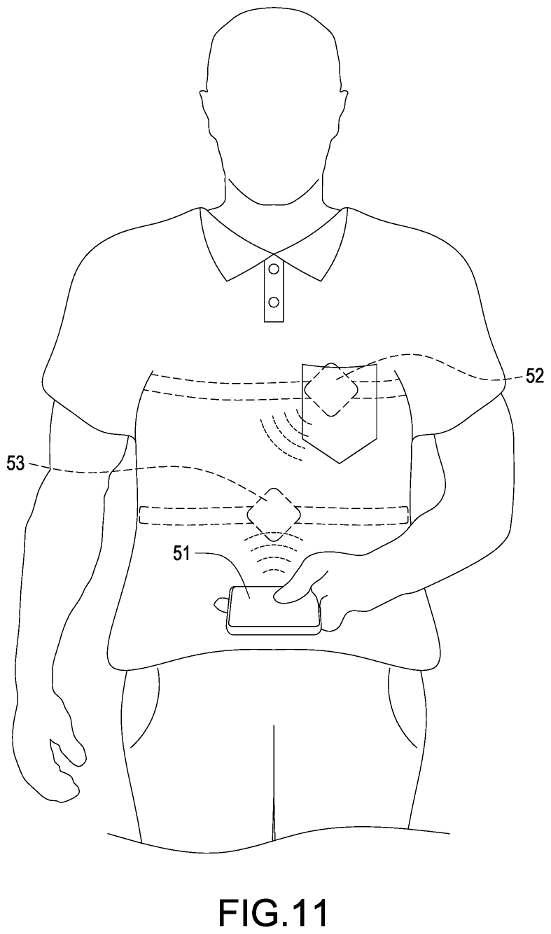

Please refer to FIGS. 8-11. FIG. 8 is a schematic view of wearing a portable circulatory shock detecting device according to one embodiment of the present disclosure, FIG. 9 is a schematic view of wearing a portable circulatory shock detecting device according to one embodiment of the present disclosure, FIG. 10 is a schematic view of wearing a portable circulatory shock detecting device according to one embodiment of the present disclosure, and FIG. 11 is a schematic view of wearing a portable circulatory shock detecting device according to one embodiment of the present disclosure.

As shown in FIG. 8, an electronic device 51 may be paired with a measurement device 50 for establishing a connection therebetween. After the connection is established, the electronic device 51 may acquire a sensed data generated by the measurement device 50. Then, a subject may put the measurement device 50 into a pocket of top clothes. The measurement device 50 may sense the vibration and the body temperature at the chest of the subject across the top clothes and generate a body surface vibration data.

Please be noted that the body surface vibration data may include the vibrations caused by walking, and these vibrations may be the noise and cause a misjudgment in the circulatory shock detection. To solve the above problem, the present disclosure may continuously analyze the body surface vibration data to obtain a number of vibration sources (such as using the waveform analysis) to determine whether the subject is in a stationary status, and start to execute the circulatory shock detection while the subject is stationary.

In one of the embodiments, as shown in FIGS. 9-11, the measurement device 52 may include a wearing structure. The wearing structure is used to make the measurement device 52 close to or touch the body surface of the subject and sense the body surface vibration data with better amplitudes and less noise.

In the embodiment shown in FIG. 9, the wearing structure includes a securing clip. The securing clip makes the measurement device 52 to be fixed in the pocket of top clothes, such that the measurement device 52 enables to be steadily close to the chest and sense the body surface vibration data of the chest surface.

In the embodiment shown in FIG. 10, the wearing structure includes a necklace chain or a collar, such that the subject may wear the measurement device 52 on the neck like a necklace. The measurement device 52 sways while the subject is moving, but the measurement device 52 becomes stationary and leans against the chest surface while the subject is stationary. Thus, the measurement device 52 may sense the body surface vibration data of the chest surface while the subject is stationary.

In the embodiment shown in FIG. 11, the wearing structure includes a fixed rope. The measurement device 52 is moored on the chest surface of the subject through the fixed rope, and stably senses the body surface vibration data at the chest surface.

In another embodiment shown in FIG. 11, a measurement device 53 is moored on the abdomen surface of the subject through the fixed rope, and stably senses the body surface vibration data at the abdomen surface.

In one of the embodiments, the wearing structure includes a patch structure. The measurement device 52 or 53 is removably pasted over the body surface of the subject through the patch structure, and stably senses the body surface vibration data at the body surface.

In one of the embodiments, the vibration-sensing module 100 may include a three-axis accelerometer configured to sense and generate a three-axis sensed acceleration data. A measurement reference surface of the measurement device is defined by the measurement device. The measurement reference surface may face to a direction of one of an X-axis, a Y-axis, and a Z-axis of the three-axis accelerometer, wherein the direction may be regarded as a reference axis. Thus, the reference axis corresponds to a vibration direction of the body surface while the measurement reference surface is parallel with the body surface, such that the body surface vibration data of the reference axis may be as the body surface vibration data.

Moreover, the monitoring module 10 may directly acquire plurality of reference axis sensed acceleration data corresponding to the measurement reference surface from the body surface vibration data (three-axis sensed acceleration data), and regard the multiple acquired single axis (reference axis) sensed acceleration data as the body surface vibration data used in the following process and analysis.

For example, the measurement reference surface faces the direction of the Z-axis of the three-axis accelerometer. Namely, the measurement reference surface is vertical with the Z-axis.

As shown in FIGS. 9-11, when the user makes the measurement reference surface of the measurement device 52/53 face to the front or back of the subject, the Z-axis direction of the measurement device 52/53 is just a vibration direction of the body surface. Namely, the measurement reference surface faces the same or opposite direction to the subject. Thus, the present disclosure may directly filter out the sensed acceleration data of X-axis and Y-axis, and save the sensed acceleration data of Z-axis as the body surface vibration data when receiving three-axis body surface vibration data (X-axis, Y-axis, and Z-axis), such as the plurality of three-axis sensed acceleration data in the embodiment. Namely, A Dimension Reduction is executed on the three-dimensional data (i.e., the three-axis body surface vibration data in the embodiment) to obtain the single-dimensional data (i.e., the sensed acceleration data of Z-axis in the embodiment).

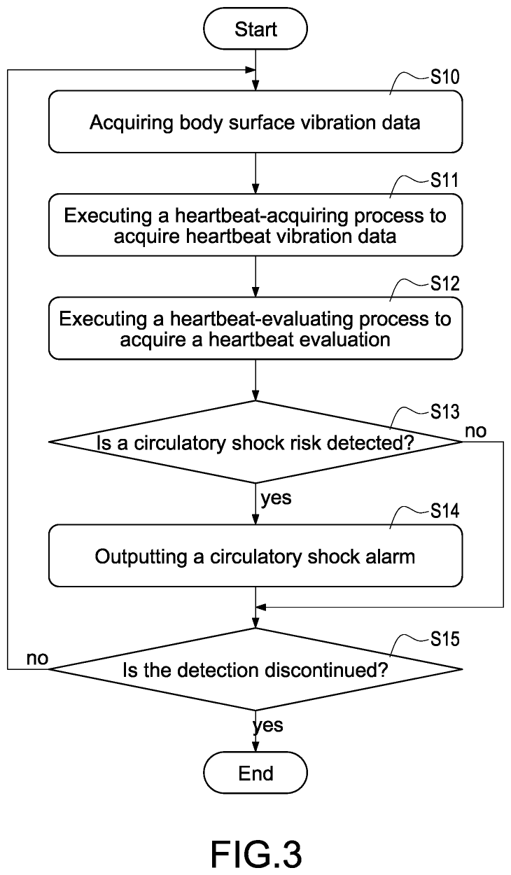

Please refer to FIG. 3, FIG. 3 is a flowchart of a method of circulatory shock monitoring according to one embodiment of the present disclosure. The method of circulatory shock monitoring of each embodiment of the present disclosure may be implemented by the portable circulatory shock detecting device of any embodiment. In the following description, the portable circulatory shock detecting device shown in FIG. 2 is mainly used to explain.

After the paring between the measurement device 10 and the electronic device 2 executing the monitoring module 11 is done, the subject may put the measurement device 10 near the body surface for starting the method of circulatory shock detection of the present disclosure.

Step S10: acquiring the body surface vibration data. The vibration-sensing module 100 of the measurement device 10 senses the vibration at the body surface to obtain the body surface vibration data of the body surface, and transmits the body surface vibration data to the communication interface 21 of the electronic device 2.

Step S11: the monitoring module 11 executes a heartbeat-acquiring process for the acquired body surface vibration data to acquire the heartbeat vibration data corresponding to the heartbeat of the subject through the heartbeat-acquiring module 30.

Step S12: the monitoring module 11 executes a heartbeat-evaluating process for the heartbeat vibration data to acquire a heartbeat evaluation through the heartbeat-evaluating module 31.

In one of the embodiments, the above heartbeat-evaluating process includes acquiring a heartbeat vibration intensity from the heartbeat vibration data, and determining whether the heartbeat is weak or stops (undetectable) as a detection reference of circulatory shock or other heart disease, such as cardiac arrest.

In one of the embodiments, the above-mentioned heartbeat evaluation may be a direct representation of the heartbeat status, for example, the heartbeat is normal, the heartbeat rate is too fast, the heartbeat rate is too slow, the heartbeat intensity is too strong, the heartbeat intensity is weak, the heartbeat stops (e.g., the heartbeat vibration is undetectable). In one of the embodiments, the heartbeat evaluation may represent either a normal heartbeat or a circulatory shock.

In one of the embodiments, considering to the detection error, the present disclosure may define a designated range consisted of continuous integers, such as one to ten, and set the two extremums of the designated range as a positive evaluation and a negative evaluation. In one embodiment, the lower the heartbeat evaluation, the more positive the heartbeat is. For example, a first heartbeat evaluation close to one may represent a higher possibility of the normal heartbeat status; a second evaluation close to ten may represent a higher possibility of the heartbeat risk (such as the circulatory shock risk). Moreover, during the above-mentioned circulatory shock detection, the present disclosure may take an index within the designated range as the heartbeat evaluation according to a trust level of the detection result. For example, the negative evaluation within six to ten is given while the heartbeat vibration becomes worse, and the positive evaluation within one to five is given while the heartbeat vibration becomes better.

Step: S13: the monitoring module 11 determines whether the determined heartbeat evaluation indicates the circulatory shock risk through the heartbeat-evaluating module 13. The circulatory shock risk may be recognized by determining whether the heartbeat intensity is zero or below an intensity threshold at which the circulatory shock may occur, the heartbeat rate is higher than a rate threshold at which the circulatory shock may occur, or the heartbeat evaluation indicates an obvious negative evaluation (such as eight to ten).

When the monitoring module 11 determines that the heartbeat evaluation indicates the circulatory shock risk, the monitoring module 11 performs a step S14. Otherwise, the monitoring module 11 performs a step S15.

Step S14: the monitoring module 11 output the circulatory shock alarm (circulatory shock alarm) through the alarm control module 35. For example, the alarm control module 35 sends a help message through the communication interface 21 or plays an alarm sound through the human-machine interface 22.

Step S15: the monitoring module 11 determines whether the circulatory shock detection is discontinued, such as the subject takes off the measurement device 10 or shuts down the monitoring module 10/monitoring module 11.

When the circulatory shock detection is discontinued, the monitoring module 11 discontinue the circulatory shock detection. Otherwise, the monitoring module 11 performs the steps S10-S15 again to continuously execute the circulatory shock detection.

The portable circulatory shock detecting device has the advantages of small size, easy to carry and low cost, etc. because of using the vibration-sensing module 100. Besides, and the portable circulatory shock detecting device may determine the heartbeat evaluation index automatically, it is suitable to be applied to implement the mobile circulatory shock monitoring.

The present disclosure may perform continuous circulatory shock detection anytime and anywhere, and actively call for help while the subject is in circulatory shock, so that the subject can get rescued immediately and the survival probability of the subject may be improved.

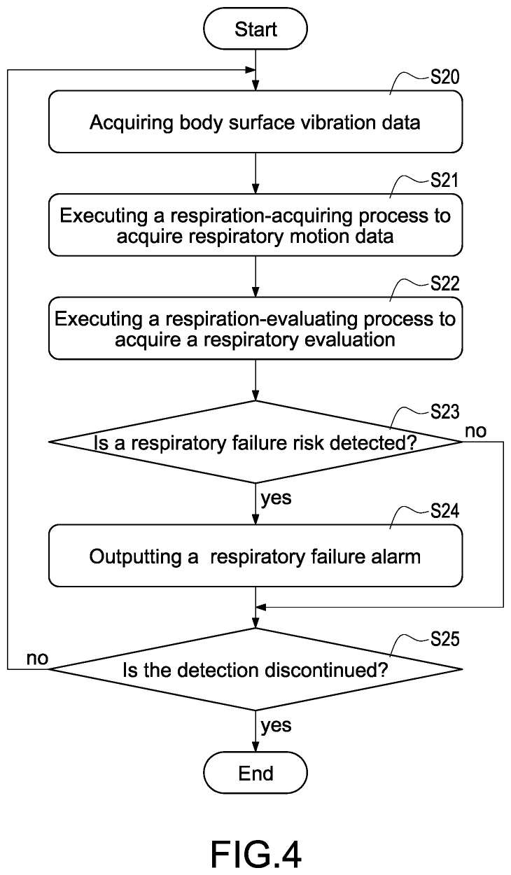

Please refer to FIGS. 3 and 4. FIG. 4 is a flowchart of respiratory evaluation according to one embodiment of the present disclosure. The method of circulatory shock monitoring may further include a respiratory evaluation function implemented by steps S20-S25 shown in FIG. 4, such that the portable circulatory shock detecting device may perform a respiratory monitoring to determine a respiratory status of the subject based on the same body surface vibration data and proactively ask for help while the respiration status is abnormal, such as a respiratory failure.

Step S20: the monitoring module 11 acquires the body surface vibration data from the measurement device 10.

Step S21: the monitoring module 11 executes a respiration-acquiring process for the body surface vibration data to acquire the respiratory motion data corresponding to the respiration through the respiration-acquiring module 32.

Step S22: the monitoring module 11 executes a respiration-evaluating process for the respiratory motion data to acquire a respiratory evaluation of the subject through the respiration-evaluating module 33.

In one of the embodiments, the above-mentioned respiration-evaluating process may acquire a respiratory intensity and a respiratory frequency from the respiratory motion data, and determine whether the respiration of the subject is rapid, weak, or discontinued as a reference for determining whether the respiratory failure or other respiratory disease occurs.

In one of the embodiments, the above-mentioned respiratory evaluation may be a direct representation of the respiratory status, for example, the respiration is normal, the respiratory rate is too fast, the respiratory rate is too slow, the respiratory intensity is too strong, the respiratory intensity is weak, the respiratory stops (e.g., the respiratory motion is undetected). In one of the embodiments, the respiratory evaluation may represent either a normal respiration or a respiratory failure.

In one of the embodiments, considering to the detection error, the present disclosure may define a designated range consisted of continuous integers, such as one to ten, and set the two extremums of the designated range are a positive evaluation and a negative evaluation. In one embodiment, the lower the respiratory evaluation, the more positive the respiratory is. For example, a first respiratory evaluation close to one may represent a higher possibility of the normal respiratory status; a second respiratory evaluation close to ten may represent a higher possibility of the respiratory risk. Moreover, during the above-mentioned respiration-evaluating process, the present disclosure may take an index within the designated range as the respiratory evaluation according to a trust level of the detection result. For example, the negative evaluation within six to ten is given while the respiratory intensity/frequency becomes worse, and the positive evaluation within one to five is given while the respiratory intensity/frequency becomes better.

Step S23: the monitoring module 11 determines whether the respiratory evaluation indicates any respiratory failure risk or other respiratory disease risks through the respiration-evaluating module 33. The risk may be recognized by determining whether the respiratory intensity is below an intensity threshold at which the respiratory failure may occur, the respiratory rate is higher than a rate threshold at which the respiratory failure may occur, or the respiratory evaluation indicates an obvious negative evaluation (such as eight to ten).

When the monitoring module 11 determines that the respiratory evaluation indicates the respiratory failure risk, the monitoring module 11 performs a step S24. Otherwise, the monitoring module 11 performs a step S25.

Step S24: the monitoring module 11 output a respiratory failure alarm through the alarm control module 35. For example, the monitoring module 11 sends a help message through the communication interface 21 or plays an alarm sound through the human-machine interface 22.

Step S25: the monitoring module 11 determines whether the respiratory monitoring is discontinued, such as the subject takes off the measurement device 10 or shuts down the monitoring module 10/monitoring module 11.

When the respiratory monitoring is discontinued, the monitoring module 11 discontinues the respiratory monitoring. Otherwise, the monitoring module 11 performs the steps S20-S25 again to continuously execute the respiratory monitoring.

The present disclosure may perform continuous respiratory monitoring anytime and anywhere, and actively call for help while the subject is in the respiratory failure, so that the subject can get rescued immediately and the survival probability may be improved.

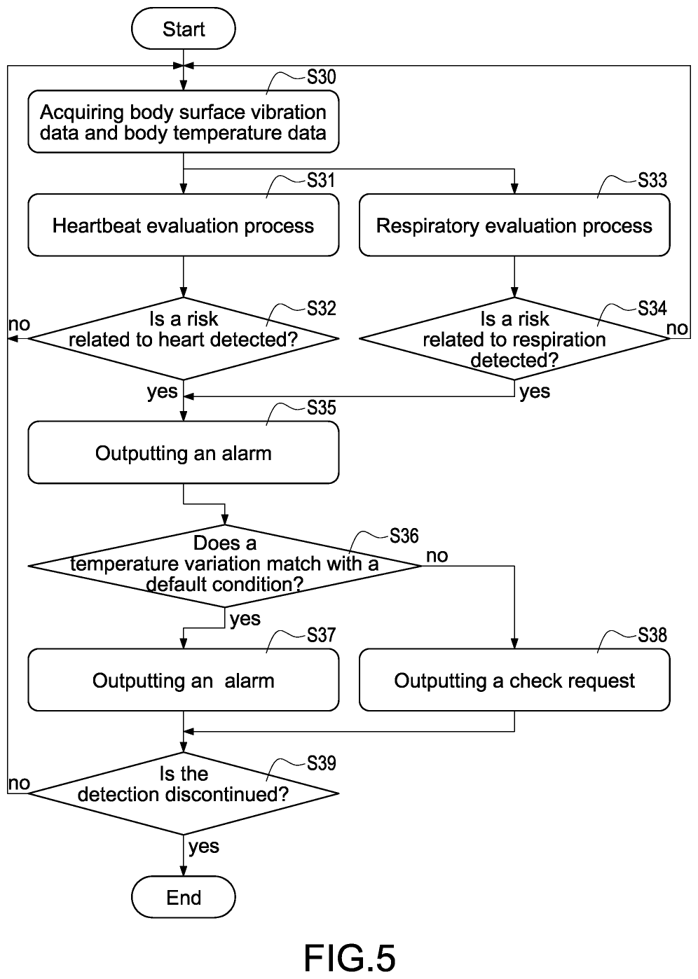

Please refer to FIGS. 3 to 5. FIG. 5 is a flowchart of heartbeat evaluation and respiratory evaluation according to one embodiment of the present disclosure. The method of circulatory shock monitoring further verifies a correctness of the heartbeat evaluation and the respiratory evaluation by referring to body temperature data.

Step S30: the monitoring module 11 acquires the body surface vibration data from the measurement device 10, and acquires the body temperature data sensed by the temperature-sensing module 103 through the temperature-acquiring module 34.

The steps S31-S32 and S35 are the same as or similar to the steps S11-S14 of FIG. 3, the relevant description is omitted for brevity.

The steps S33-S34 and S35 are the same as or similar to the steps S21-S24 of FIG. 4, the relevant description is omitted for brevity.

In one of the embodiments, in the steps S31, S32 and S35, the monitoring module 11 may compute another heartbeat evaluation for indicating other heartbeat risk based on the heartbeat vibration data, determine whether the computed heartbeat evaluation indicates the corresponding risk, and issue a corresponding alarm when the risk is detected. The other heartbeat risk mentioned above may include a risk of cardiac arrest, a risk of fast heartbeat, a risk of slow heartbeat, etc.

Taking the risk of cardiac arrest for example, the heartbeat evaluation is determined to indicate the risk of cardiac arrest after a heartbeat intensity of the heartbeat vibration data is undetectable for a default heartbeat monitoring time, and a cardiac arrest alarm is outputted.

Taking the risk of fast heartbeat or the slow heartbeat for example, the heartbeat evaluation is determined to indicate the risk of fast heartbeat or slow heartbeat after a heartbeat rate of the heartbeat vibration data is higher than a default heartbeat rate upper limit or lower than a default heartbeat rate lower limit for the default heartbeat monitoring time, and a fast heartbeat alarm or a slow heartbeat alarm is outputted.

In one of the embodiments, in the steps S33, S34 and S35, the monitoring module 11 may compute another respiratory evaluation for indicating other respiratory risk based on the respiratory motion data, determine whether the computed respiratory evaluation indicates the corresponding risk, and issue a corresponding alarm when the risk is detected. The other respiratory risk mentioned above may include a risk of apnea, a risk of deep respiratory, a risk of shallow respiratory, etc.

Taking the risk of apnea for example, the respiratory evaluation is determined to indicate the risk of apnea after a respiratory intensity of the respiratory motion data is undetectable for a default respiratory monitoring time, and a respiratory stop alarm is outputted.

Taking the risk of deep respiratory/shallow respiratory for example, the respiratory evaluation is determined to indicate the risk of deep respiratory or shallow respiratory after a respiratory amplitude of the respiratory motion data is higher than a default respiratory amplitude rate upper limit or lower than a default respiratory amplitude lower limit for the default respiratory monitoring time, and a deep respiratory alarm or a shallow respiratory alarm is outputted.

When any risk is detected, the step S36 is performed: the monitoring module 11 determines whether a variation of the body temperature data matches with the conditions corresponding to each of the risks. For example, when the circulatory shock occurs, the lower body temperature usually happens on the subject. When the respiratory failure occurs, the higher body temperature usually happens on the subject. The above conditions may include an unnaturally rising body temperature or an unnaturally falling body temperature.

In one of the embodiments, the present disclosure may generate the heartbeat evaluation and the respiratory evaluation based on a comprehensive reference of the heartbeat vibration data, the respiratory motion data, and the body temperature data.

When the variation of the body temperature data matches with the corresponding condition, the monitoring module 11 performs a step S37. Otherwise, the monitoring module 11 performs a step S38.

Step S37: the monitoring module 11 outputs a corresponding alarm through the alarm control module 35.

Step S38: the monitoring module 11 outputs a check request to ask the subject to check whether the outputted alarm is true or a misjudgment through the human-machine interface 22, and instantly stops or modifies the alarm outputted in the step S35 after the alarm is determined as the misjudgment.

In one of the embodiments, the step S35 may be modified to output the alarm only if no response to the check request is received from the subject over a default time (such as the subject fainted). Namely, a step similar to outputting the check request of the step S38 is performed after a determination of the step S32 or the step S34 is “yes” and before performing the step S35, and the step S35 is performed to output the alarm after no response for the check request is received. Besides, the step S36 may be simultaneously performed to evaluate the risk by considering the body temperature data after no response for the check request is received.

Step S39: the monitoring module 11 determines whether the heartbeat monitoring and the respiratory monitoring are discontinued, for example, the subject takes off the measurement device 10 or shuts down the monitoring module 10/monitoring module 11.

When the monitoring is discontinued, the method is discontinued. Otherwise, the monitoring module 11 performs the steps S30-S39 again to continuously execute the monitoring.

As a result, the present disclosure may effectively improve an accuracy of detection and a correctness of alarms by referring to the body temperature data, and may be used to detect more types of heartbeat risks and respiratory risks.

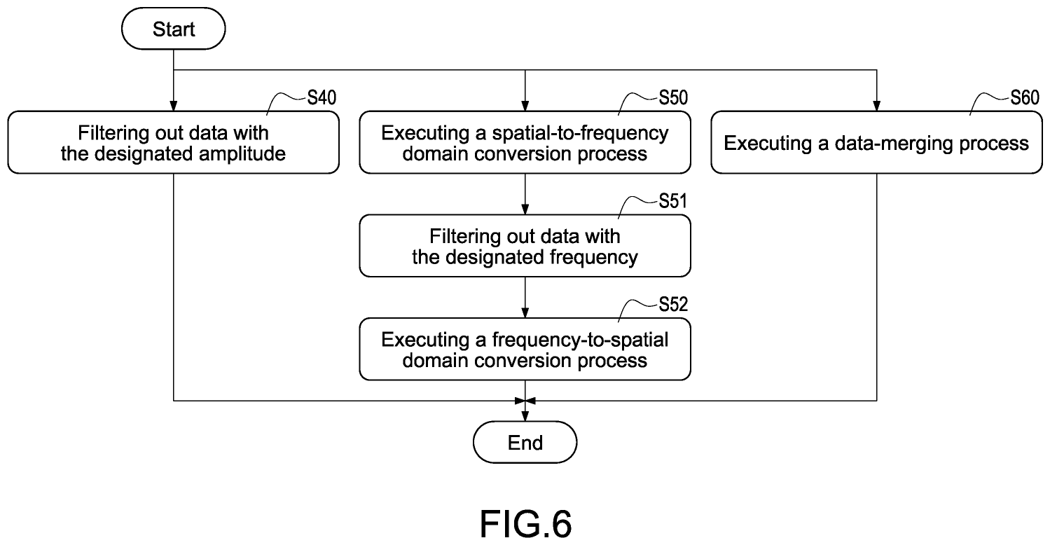

Please refer to FIGS. 3 to 6. FIG. 6 is a flowchart of data process according to one embodiment of the present disclosure. In this embodiment, the present disclosure provides at least three data processing methods to filter out the noise and/or recognize the heartbeat vibration data and the respiratory motion data from the body surface vibration data. The data processing methods include threshold filtering, domain conversion and data merging.

The threshold filtering includes a step S40.

Step S40: the monitoring module 11 executes a noise-filtering process for the body surface vibration data to filter out the noise through the data-filtering module 36.

In one of the embodiments, the monitoring module 11 filters out the data with an amplitude less than a default noise threshold from the body surface vibration data.

In one of the embodiments, the monitoring module 11 computes a difference of adjacent data values of the body surface vibration data, and filters out data with the difference from adjacent data less than a difference threshold, such as deleting one or more adjacent data value(s) corresponding to the difference.

In one of the embodiments, the monitoring module 11 may execute the above noise-filtering process for the heartbeat vibration data and the respiratory motion data to filter out the noise in the heartbeat vibration data and the respiratory motion data. During the above noise-filtering process, different noise thresholds may be used for the different data. For example, a higher noise threshold is used for a heartbeat-related process because the heartbeat vibration changes sharply, and a lower noise threshold is used for a respiration-related process because the respiratory motion changes slowly, but this specific example is not intended to limit the scope of the present disclosure.

In one of the embodiments, the monitoring module 11 may define a default heartbeat amplitude range or a default respiratory amplitude range through the data-filtering module 36, and acquire the heartbeat vibration data with the amplitudes within the heartbeat amplitude range and the respiratory motion data with the amplitudes within the respiratory amplitude range from the body surface vibration data.

The domain conversion includes steps S50-S52.

Step S50: the monitoring module 11 executes a spatial-to-frequency domain conversion process (such as Fast Fourier transform, Discrete Wavelet Transform, etc.) for the body surface vibration data to obtain a body surface frequency data through the spatial-frequency domain conversion module 37.

Step S51: the monitoring module 11 acquires a partial frequency band of the body frequency data from the body surface frequency data based on a default frequency.

For example, in general, the respiratory rate of adults is 10-20 times per minute (referred to as a default respiratory frequency), and the heartbeat rate of adults is 60-100 times per minute (referred to as a default heartbeat frequency). The monitoring module 11 may capture the heartbeat frequency data and respiratory frequency data within the default respiratory frequency and the default heartbeat frequency.

Step S52: the monitoring module 11 executes a frequency-to-spatial domain conversion process for the body surface frequency data (i.e., the heartbeat frequency data or the respiration frequency data) to acquire the heartbeat vibration data and/or the respiratory motion data in the spatial domain through the spatial-frequency domain conversion module 37.

In one of the embodiments, the monitoring module 11 may subtract the body surface vibration data from the heartbeat vibration data to obtain the respiratory motion data after acquiring the heartbeat vibration data.

In one of the embodiments, the monitoring module 11 may subtract the respiratory motion data from the body surface vibration data to obtain the heartbeat vibration data after acquiring the respiratory motion data.

The above-mentioned heartbeat-acquiring process may include filtering out a partial data not belonging to the heartbeat from the body surface vibration data to generate the heartbeat vibration data.

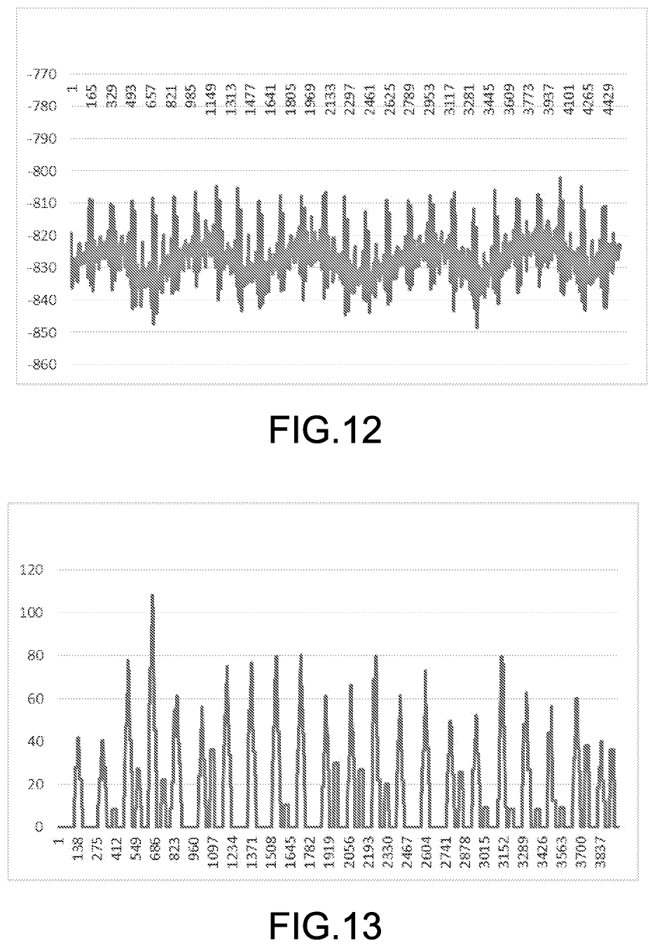

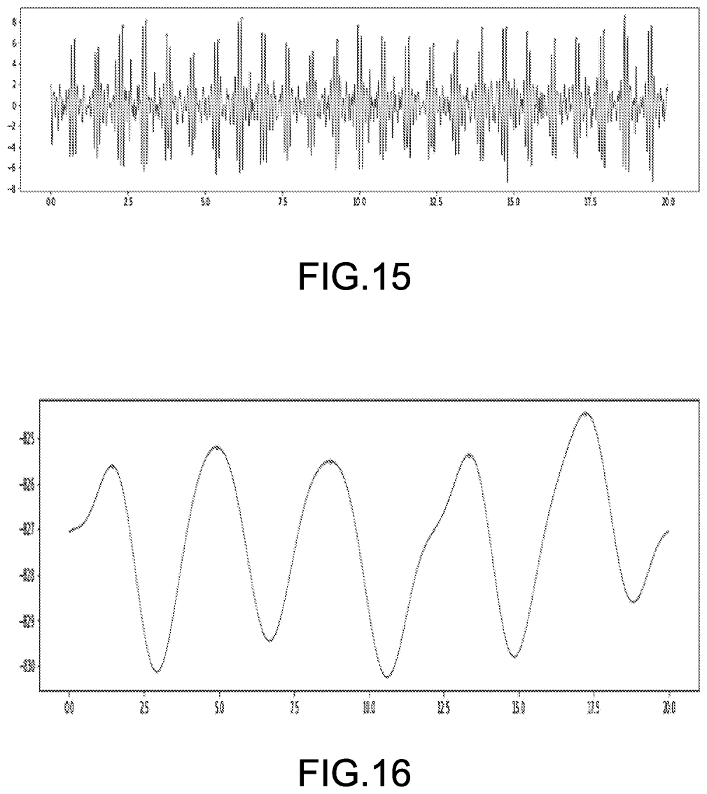

Please refer to FIGS. 12, 15 and 16. FIG. 15 is a waveform graph of heartbeat vibration data acquired from body surface vibration data of FIG. 12 according to one embodiment of the present disclosure, and FIG. 16 is a waveform graph of respiratory motion data acquired from body surface vibration data of FIG. 12 according to one embodiment of the present disclosure.

FIG. 12 shows the body surface vibration data sensed by the measurement device 10. The vibration sources in the body surface vibration data include the heartbeat vibration, the respiratory motion, and other vibrations (noises).

The heartbeat-acquiring module 30 of the present disclosure may transform the body surface vibration data in the spatial domain into the body surface frequency data, and derive the heartbeat frequency data from the body surface frequency data based on the default heartbeat frequency. Finally, the heartbeat-acquiring module 30 may transform the heartbeat frequency data into the heartbeat vibration data in the spatial domain as shown in FIG. 15.

Moreover, the respiration-acquiring module 32 of the present disclosure may derive the respiratory frequency data from the body surface frequency data based on the default respiratory frequency. Finally, the respiration-acquiring module 32 may transform the respiratory frequency data into the respiratory motion data in the spatial domain as shown in FIG. 16.

In one of the embodiments, the monitoring module 11 of the present disclosure may execute the analysis process for the heartbeat vibration data (FIG. 15) and the respiratory motion data (FIG. 16) to obtain a corresponding heartbeat waveform and a corresponding respiratory waveform. The above waveforms may be used to detect heartbeat-related diseases or respiration-related diseases instantly, such as the circulatory shock, the cardiac arrest, the respiratory failure, the apnea, etc.

The data merging includes a step S60.

Step S60: the monitoring module 11 executes a data-merging process for the body surface vibration data (such as the heartbeat vibration data or the respiratory motion data) to acquire a simplified data.

In one of the embodiments, the monitoring module 11 executes the data-merging process for the heartbeat vibration data based on a default mergence interval to acquire a heartbeat mergence data, and executes the above-mentioned heartbeat-evaluating process for the heartbeat mergence data. The default mergence interval may be determined based on a sampling rate and/or a heartbeat rate. For example, the heartbeat mergence interval may be 25 or any value within 25-30, but this specific example is not intended to limit the scope of the present disclosure. Each data value of the above heartbeat mergence data is respectively generated based on a plurality of data values of the heartbeat vibration data in each heartbeat mergence interval.

In one of the embodiments, in the circulatory shock detection, the heartbeat evaluation is assessed as the circulatory shock risk when the heartbeat mergence data shows that a heartbeat intensity weakens and/or a heartbeat rate is too high or too low (such as the heartbeat rate is higher than a heartbeat rate upper limit or lower than a heartbeat rate lower limit) for a default heartbeat monitoring time (such as five seconds or ten seconds).

In one of the embodiments, in the circulatory shock detection, the heartbeat evaluation is assessed as the risk of cardiac arrest when the heartbeat mergence data shows that the heartbeat intensity is undetected (such as no heartbeat or weak heartbeat) for the default heartbeat monitoring time.

In one of the embodiments, the monitoring module 11 executes a data-merging process based on a default mergence interval (referred to as a respiratory mergence interval) to acquire a respiratory mergence data, and execute the respiratory-evaluating process for the respiratory mergence data. The respiratory mergence interval may be determined based on a sampling rate and/or a respiratory rate, such as 300 or any value within 200-400, but this specific example is not intended to limit the scope of the present disclosure. Each data value of the respiratory mergence data is respectively computed (such as accumulate or average) based on a plurality of data values of the respiratory motion data in each respiratory mergence interval.

In one of the embodiments, in the respiration-evaluating process, the respiratory evaluation is assessed as the respiratory failure risk when the respiratory mergence data shows that a respiratory intensity weakens and/or a respiratory rate is too high or too low (such as the respiratory rate is higher than a respiratory rate upper limit or lower than a respiratory rate lower limit) for a default respiratory monitoring time (such as sixty seconds or ninety seconds).

In one of the embodiments, in the respiration-evaluating process, the respiratory evaluation is assessed as the risk of apnea when the respiratory mergence data shows that the respiratory intensity is undetected (such as no respiration or weak respiration) for the default respiratory monitoring time.

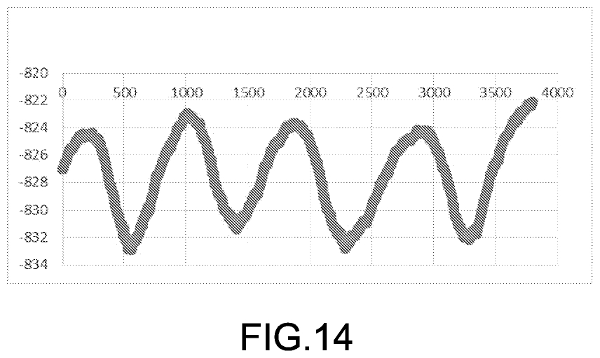

Please refer to FIGS. 12 to 14. FIG. 12 is a waveform graph of body surface vibration data according to one embodiment of the present disclosure, FIG. 13 is a waveform graph of heartbeat vibration data acquired from body surface vibration data of FIG. 12 according to one embodiment of the present disclosure, and FIG. 14 is a waveform graph of respiratory motion data acquired from body surface vibration data of FIG. 12 according to one embodiment of the present disclosure. FIGS. 13 and 14 are related to using the data-merging process algorithm to derive the heartbeat vibration data and the respiratory motion data from the body surface vibration data.

FIG. 12 shows the body surface vibration data sensed by the measurement device 10. The vibration sources in the body surface vibration data include the heartbeat vibration, the respiratory motion, and other vibrations (noises).

In order to correctly execute the circulatory shock detection and accurately obtain the heartbeat evaluation, the monitoring module 11 in this embodiment computes an amplitude difference data of every two adjacent information values of the body surface vibration data. For example, the amplitude difference data may comprise a data of an amplitude difference between a first data value and a second data value, a data of an amplitude difference between the second data value and a third data value, and so on.

Then, the monitoring module 11 deletes the data of the amplitude difference that is less than a noise threshold (such as eight) from the amplitude difference data to filter the amplitude difference data.

Then, the filtered amplitude difference data is processed. The monitoring module 11 accumulates continuous amplitude difference data of the filtered amplitude difference data in each designated data interval (e.g., in each heartbeat mergence interval which is 25) to obtain the heartbeat mergence data as the heartbeat vibration data. Each designated data interval may have the same length and be obtained by shifting a fixed value for the last designated data interval. For example, a first data value of the heartbeat mergence data is computed by accumulating a first data value to a twenty-fifth data value of the continuous amplitude difference data, a second data value of the heartbeat mergence data is computed by accumulating a second data value to a twenty-sixth data value of the continuous amplitude difference data, and so on.

Thus, this embodiment may obtain the heartbeat intensity and heartbeat rate clearly by analyzing the waveform of the heartbeat vibration data, and enable to detect the circulatory shock and other heart diseases instantly.

Moreover, in order to accurately obtain the respiratory evaluation, in one embodiment, an average of a plurality of adjacent data values in each designated interval (e.g., in each respiratory mergence interval which is 300) of the body surface vibration data is calculated to obtain the respiratory mergence data as the respiratory motion data. Each designated data interval may have the same length and be obtained by shifting a fixed value for the last designated data interval. For example, a first data value of the respiratory mergence data is computed by calculating an average of a first data value to a 300th data value of the plurality of adjacent data values, a second data value of the respiratory mergence data is computed by calculating an average of a second data value to a 301st data value of the plurality of adjacent data values, and so on.

Thus, this embodiment may obtain the respiratory intensity and respiratory rate clearly by analyzing the waveform of the respiratory motion data, and enable to detect the respiratory failure and other respiratory diseases instantly.

While this disclosure has been described by means of specific embodiments, numerous modifications and variations could be made thereto by those skilled in the art without departing from the scope and spirit of this disclosure set forth in the claims.

What is claimed is:

1. A portable circulatory shock detecting device, comprising:
a measurement device, comprising:
a vibration-sensing module, configured to sense a body surface vibration data of a subject; and
a communication module, electrically connected to the vibration-sensing module and configured to transmit the body surface vibration data to an electronic device; and
a monitoring module, configured to control the electronic device to execute a heartbeat-acquiring process for the body surface vibration data to acquire a heartbeat vibration data corresponding to a heartbeat of the subject, control the electronic device to execute a heartbeat-evaluating process for the heartbeat vibration data to generate a heartbeat evaluation of the subject according to the heartbeat vibration data, and control the electronic device to output a circulatory shock alarm after the heartbeat evaluation is assessed as a circulatory shock risk.

2. The portable circulatory shock detecting device according to claim 1, wherein the measurement device comprises a temperature-sensing module electrically connected to the communication module and configured to sense a body temperature data of the subject;
wherein the monitoring module is configured to control the electronic device to output the circulatory shock alarm after the heartbeat evaluation is assessed as the circulatory shock risk and a variation of the body temperature data matches with a circulatory shock variation.

3. The portable circulatory shock detecting device according to claim 1, wherein the monitoring module is configured to execute a noise-filtering process for the body surface vibration data to generated a filtered body surface vibration data by filtering out a data with an amplitude lower than a noise threshold or a data with a difference from adjacent data values less than a difference threshold, and execute the heartbeat-acquiring process for the filtered body surface vibration data.

4. The portable circulatory shock detecting device according to claim 1, wherein the monitoring module is configured to execute a data-merging process based on plurality of heartbeat mergence intervals to acquire a heartbeat mergence data, and execute the heartbeat-evaluating process for the heartbeat mergence data;
wherein each data value of the heartbeat mergence data is respectively computed based on a plurality of data values of the heartbeat vibration data in each heartbeat mergence interval.

5. The portable circulatory shock detecting device according to claim 1, wherein the monitoring module is configured to execute a spatial-to-frequency domain conversion process for the body surface vibration data to acquire a body surface frequency data, acquire a heartbeat frequency data based on a default heartbeat frequency from the body surface frequency data, and execute a frequency-to-spatial domain conversion process for the heartbeat frequency data to acquire the heartbeat vibration data.

6. The portable circulatory shock detecting device according to claim 1, wherein the monitoring module is configured to determine the heartbeat evaluation indicating the circulatory shock risk after a heartbeat intensity of the heartbeat vibration data weakens for a heartbeat monitoring time, or a heartbeat rate of the heartbeat vibration data remains higher than a heartbeat rate upper limit or lower than a heartbeat rate lower limit for the heartbeat monitoring time.

7. The portable circulatory shock detecting device according to claim 1, wherein the monitoring module is configured to execute the heartbeat evaluation process based on the heartbeat vibration data and at least one of a body temperature data and a respiratory motion data to determine the heartbeat evaluation.

8. The portable circulatory shock detecting device according to claim 1, wherein the monitoring module is configured to determine the heartbeat evaluation indicating a heartbeat stop risk after a heartbeat intensity of the heartbeat vibration data is undetectable for a heartbeat monitoring time;
wherein the monitoring module is configured to control the electronic device to output a heat stop alarm after the heartbeat evaluation indicates the heartbeat stop risk.

9. The portable circulatory shock detecting device according to claim 1, wherein the monitoring module is configured to execute a respiration-acquiring process for the body surface vibration data to acquire a respiratory motion data corresponding to a respiratory activity of the subject, execute a respiration-evaluating process for the respiratory motion data to acquire a respiratory evaluation of the subject, and control the electronic device to output a respiratory failure alarm after the respiratory evaluation is assessed as a respiratory failure risk.

10. The portable circulatory shock detecting device according to claim 9, wherein the monitoring module is configured to execute a data-merging process based on plurality of respiratory mergence intervals to acquire a respiratory mergence data, and execute the respiration-evaluating process for the respiratory mergence data;
wherein each data value of the respiratory mergence data is respectively computed based on a plurality of data values of the respiratory motion data in each respiratory mergence interval.

11. The portable circulatory shock detecting device according to claim 9, wherein the monitoring module is configured to execute the respiration-evaluating process based on the heartbeat vibration data, the respiratory motion data, and a body temperature data to determine the respiratory evaluation.

12. The portable circulatory shock detecting device according to claim 9, wherein the monitoring module is configured to execute a spatial-to-frequency domain conversion process for the body surface vibration data to acquire a body surface frequency data, acquire a respiratory frequency data from the body surface frequency data based on a default respiratory frequency, and execute a frequency-to-spatial domain conversion process for the respiratory frequency data to acquire the respiratory motion data.

13. The portable circulatory shock detecting device according to claim 9, wherein the monitoring module is configured to determine the respiratory evaluation indicating the respiratory failure risk after a respiratory intensity of the respiratory motion data weakens for a respiratory monitoring time, or a respiratory rate of the respiratory motion data remains higher than a respiratory rate upper limit or lower than a respiratory rate lower limit for the respiratory monitoring time.

14. The portable circulatory shock detecting device according to claim 1, wherein the monitoring module is configured to determine the respiratory evaluation indicating a respiratory stop risk after a respiratory intensity of the heartbeat vibration data is undetectable for a respiratory monitoring time;
wherein the monitoring module is configured to control the electronic device to output a respiratory stop alarm after the respiratory evaluation indicates the respiratory stop risk.

15. The portable circulatory shock detecting device according to claim 1, wherein the measurement device comprises:
a wearing structure, making the measurement device to touch a body surface of the subject and making a measurement reference surface of the measurement device to face to same or opposite direction as the subject;
wherein the measurement reference surface faces to a direction of an X axis, a Y axis, or a Z axis of the vibration-sensing module.

16. The portable circulatory shock detecting device according to claim 15, wherein the vibration-sensing module comprises at least one accelerometer to sense and generate a sensed acceleration data;
wherein the monitoring module is configured to acquire plurality of the sensed acceleration data from the body surface vibration data as the body surface vibration data to be processed, wherein the acquired sensed acceleration data are corresponding to a reference axis corresponding to the measurement reference surface.

17. The portable circulatory shock detecting device according to claim 1, wherein the communication module comprises a Bluetooth low power interface;
wherein the measurement device comprises:
a battery configured to store electricity and provide electricity; and
an indication module configured to indicate a status of the measurement device.
